(19) United States
(12) Patent Application Publication
(10) Pub. No.: US 2006/0042990 A1
(43) Pub. Date: Mar. 2, 2006

(54) Medication kit

(76) Inventor: Jerry H. Galuten, Riverdale, NY (US)

Correspondence Address: Gregor N. Neff, Esq.;c/o Kramer Levin Naftalis & Frankel LLP, 919 Third Avenue, New York, NY, 10022, US

(21) Appl. No.: 10/925,187

(22) Filed: Aug. 24, 2004

(51) Int. Cl. B65D 69/00; A61B 19/02

(52) U.S. Cl. 206/570; 206/440

(57) ABSTRACT

The medication kit includes a bottle of water, a support card to which the bottle is secured, and a package of pills to be taken with the water, all wrapped in a cover. Alternatively, the kit includes self-adhesive bandages, a support card to which a bottle of wound cleansing liquid and/or a tube of anti-bacterial material secured, with a clear covering. Preferably, each bottle or tube is fitted into a shaped opening in the card to help hold it in place.

The present invention relates to medication kits, specifically to medication kits containing limited quantities of two or more medication products to be used together.

It long has been a problem to carry and use medications when traveling. Although one can carry aspirin or other analgesics one's pocket or purse, capsules or tablets usually require that they be taken with water to wash them down. Therefore, the person taking the medication must look for a source of drinking water, or carry a bottle of water with him or her. The same problem exist for oral prescription drugs, vitamins or other such items which must be taken while one is traveling.

Similar problems exist with the carrying of other medications, such as self-adhesive bandages while traveling. Although it is easy enough to carry the bandages, usually it is highly desirable or necessary to cleanse a wound to clean out the dirt and disinfect the wound before bandaging it. Also, it is very desirable to apply an anti-bacterial cream or liquid to the wound before bandaging it.

In order to serve these needs, a person might be required carry a bottle of hydrogen peroxide or alcohol, and a separate container of anti-bacterial cream or liquid, as well as bandages.

It has been proposed in the past to alleviate the foregoing problems in taking oral medications by providing a combined package containing tablets to be swallowed and a plastic packet of water for washing the medication down.

These prior attempts have been less than fully satisfactory because of their cost, and because the proposed products have been difficult to use.

Accordingly, it is an object of present invention to provide a medication kit which alleviates or solves foregoing problems.

In particular, it is an object to provide a medication kit which is relatively simple and inexpensive to manufacture and easy to use, while being relatively compact and sturdy so as to be easy to carry and relatively inexpensive to pack and ship.

In accordance with the present invention, these objectives are met by the provision of a medication kit for oral medication in which a bottle of water with a package of medication is package together in a single container.

Preferably, a support card is provided and a transparent plastic cover is applied to the card and the products to protect them and hold them in place.

A further preferred feature is to provide a shaped opening in the card into which the bottle fits so that it extends through the card and is supported and held stable in one position by the edges of the opening and the plastic cover.

Preferably, the card is coated on at least one surface with thermoplastic material so that the plastic cover can be secured to the card by the application of heat.

Preferably, the bottle of water is a small plastic bottle with a relatively wide mouth which is easy to drink out of. The bottle is commonly manufacturer in large quantities and at low cost for other purposes, and thus is relatively plentiful and inexpensive.

The kit is relatively compact so that it easily fits into a purse, a traveling toiletries kit, a fishing tackle box, a coat or pants pocket or an automobile glove compartment, or in a household medicine cabinet, etc.

Another medication kit advantageously provides self-adhesive bandages together with bottle of hydrogen peroxide or alcohol for cleaning a wound, and/or a tube of anti-bacterial cream or liquid. Optionally, swabs can be included for use in cleaning a wound with the cleansing liquids.

Again, the invention takes advantage of the widespread availability of each of the items separately, but fasten them securely to a card in a protective package which is compact convenient and easy to use.

The foregoing and other objects and advantages of the invention will be setforth in or apparent from following description and drawings.

### IN THE DRAWINGS

FIG. 1 is a perspective view of the preferred medication kit provided by the present invention;

FIG. 2 is a front elevation view a component of the kit shown in FIG. 1;

FIG. 3 is a left side elevation view of the kit shown in FIG. 1;

FIG. 4 is a perspective view of another medication kit provided by the present invention; and

FIG. 5 is a perspective view of a further medication kit provided by the present invention.

### ORAL MEDICATION KIT

FIG. 1 shows the preferred form of a novel oral medication kit 10. The kit 10 includes a support card 12, a bottle of water 14, and a package of medication 16. In this case, the package 16 contains two analgesic tablets 22, such as aspirin tablets or the equivalent. However, the medication can be any type of capsules, tablets or other form of medication taken orally. The medication can be over-the-counter or prescription drugs, vitamins, antacids or other orally ingested items. The package 16 also could be a second bottle of liquid to be mixed or taken with the water in the bottle 14.

The bottle 14 preferably is a plastic bottle, with a main body 17 and a screw top 15 covering a relatively wide mouth. Preferably, the bottle 14 contains certified spring water (certified by the states in which it is sold), sterilized water, or any other type of pure, sanitary water.

The plastic bottle 14 is made of a material such as PET. In a preferred embodiment, the bottle 14 has a height H (see FIG. 1) of three and one quarter inches, a width W of two inches and thickness T (FIG. 3) of approximately one inch. The mouth of the bottle is approximately one half inch in diameter, which is relatively large so as to enable a person to drink easily out of the bottle. Such bottles currently are manufactured in substantial quantities for use as small bottles of mouthwash.

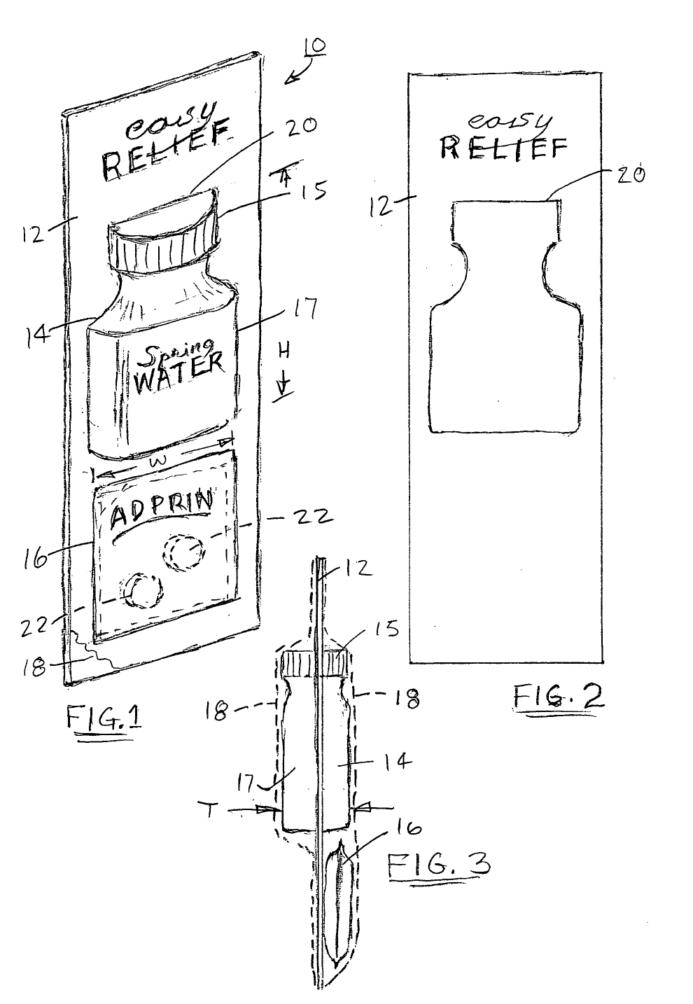

Referring to FIG. 2, the card 12 has an opening 20 in the shape of the bottle 14, with dimensions the same as or just slightly larger than that of the bottle. The bottle is fitted into the hole 20 so that it extends completely through the card 12 and extends out from both the front surface and the back surface, as it is shown FIG. 3 of the drawings. Preferably, the bottle 14 fits into the opening 20 with a slight friction fit so as to help hold it in place.

In the case of aspirin or other analgesics, the package 16 preferably compromises an envelope sealed around the edges and made of plastic-coated aluminum foil for protecting the medication inside and giving the product a relatively long shelf life. Analgesics are sold in such envelopes at the present time.

Because of the purity of the water in the bottle 14, it also has a long shelf life. As result, the kit has a relatively long shelf life and can be carried in a purse or other travel container unused for a relatively long period of time without loss of effectiveness.

The combination of the support card 12 with the water bottle 14 fitted into the opening 20 and the medication package 16 is encased in a transparent plastic enclosure 18 as it is shown in FIG. 3 and indicated schematically in FIG. 1.

Preferably, the card 12 is coated on one or both of its two broad surfaces with a thermoplastic material so that the thermoplastic cover 18 is attached to the surface of the card by heat.

Preferably, the enclosure 18 is made of relatively stiff plastic material which is vacuum or pressure-formed into the appropriate shape, and is then positioned around the card 12, bottle 14 and medicine package 16, and heat-bonded to the card. The resulting package is a double-sealed bottle of water and medication package which is compact, sturdy, relatively inexpensive and easy to use.

The provision of a relatively large quantity of water (e.g., 1½ to 1¾ ounces) in the bottle 14 causes a special problem due to the weight of the container and its contends. This weight might threaten the integrity of the finished package, but for the use of the opening 20 to help hold the bottle securely in place without exerting excess strain on the covering material 18.

The mounting of the bottle has further advantages. For example, by causing approximately one half of the bottle to extend outwardly from each of the two broad surfaces of the card, the bottle extends outwardly to almost the same distance as the package 16. This helps minimize the thickness of the kit so that more of the kits can be package in a given volume for shipping, thus reducing shipping costs.

### Cut and Abrasion Kit

FIG. 4 shows the first embodiment 30 of a cut and abrasion kit constructed in accordance with the present invention.

The kit 30 includes a support card 32, a bottle 34 of wound cleansing liquid such hydrogen peroxide or alcohol, and some self-adhesive bandages 38 and 40, e.g., one large bandage 38 and a smaller bandage 40.

Optionally, one or more cotton swabs 36 are provided for use in cleansing a wound with the liquid in the bottle 34.

Preferably, the bottle 34 is fitted into an opening 42 similar the opening 20 shown FIG. 1 and 2 so that the opposite side of the bottle 34 extends through the opposite side of the card. Of course, a covering indicated schematically at 39 of transparent plastic material similar to the covering 18 shown FIG. 3 is provided to cover the front and back side of the card 32 the components of the kit.

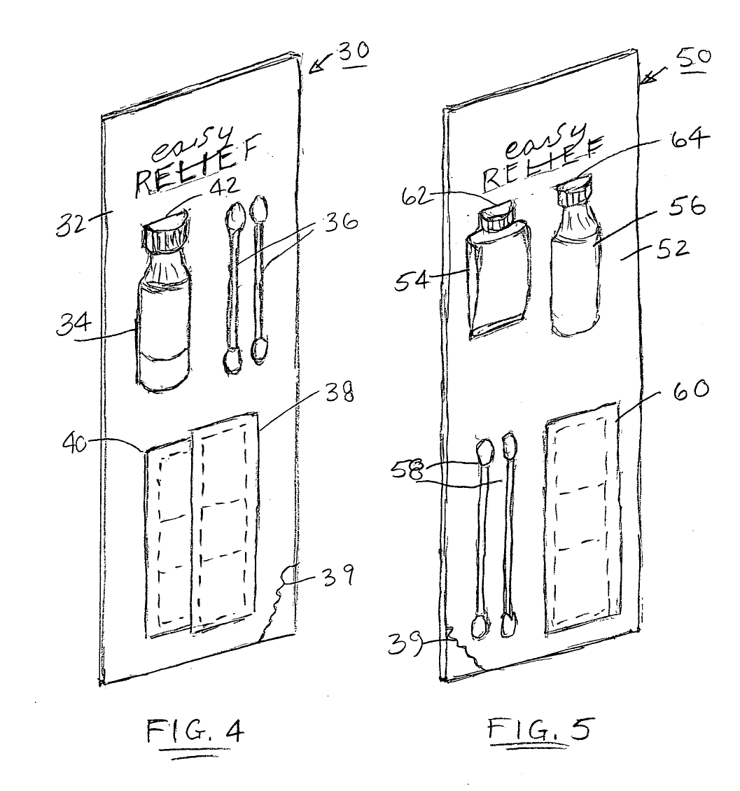

FIG. 5 shows another version 50 of the cut and abrasion kit. The kit includes a support card 52, one or more bandages 60, optional swabs 58, a tube of anti-bacterial cream or gel such as “Neosporin” or the like, and a bottle 56 of cleansing liquid such as hydrogen peroxide or alcohol.

The tube 54 is fitted into a shaped opening 62, and the bottle 56 is fitted into another shaped opening 64. A transparent plastic covering 39 covers both sides of the card 52 and is secured to the thermoplastic surfaces of the card by heating sealing. (“Heating sealing” as used herein, includes ultrasonic bonding).

The kits 30 and 50 shown FIGS. 4 and 5 have many of the advantages enjoyed by the kit 10 shown in FIGS. 1-3. The bottle of liquid in each kit is held securely in place by being fitted into a shaped opening in a support card. The pre-packaged bandages are commonly available at relatively low cost. The same is true for the swabs 36 or 58, and the other kit components.

Preferably, the bottles 34 and 56 are relatively small, as is the tube of antiseptic cream 54. This is especially advantageous in that most people seldom use these substances completely from a large bottle or tube before the marked expiration dates. Thus, much of the materials normally goes to waste. It is easier to use up the contents of one of the small bottles or tubes before the expiration date, thus avoiding same wastage and encouraging the use of fresher medications.

The kit 30 can be used to first cleanse a wound, such as a scraped knee or similar abrasion, by use of the cleansing material in the bottle 34 and the cotton swabs 36, before bandaging the wound.

The kit 50 gives more options in that the wound can be cleaned by use of the bottled cleansing material and a swab, and then the antiseptic cream can be applied before the wound is bandaged.

Each of the kits can be made inexpensively enough so that the ordinary householder can use it at home as well as during travels. Instead of buying a large container of bandages, a large tube of antiseptic cream, and a large bottle of hydrogen peroxide or alcohol, the homeowner can buy one or two relatively inexpensive kits for the occasional needs of the household and replace the kit at relatively low cost without excess wastage of materials.

It should be understood that the present invention can be practiced in a variety of different forms other than those specifically described above. Therefore, it is to be understood that the protection of this patent application is limited only by the claims set forth herein below.

1. A medication kit comprising, in combination, a package, and a combination of two or more medication products in said package, said products being selected from the group consisting of:
(a) a bottle of water with a container of oral medication materials to be swallowed with said water;
(b) at least one self-adhesive bandage with a bottle of disinfecting wound cleaner;
(c) at least one self-adhesive bandage with a container of anti-bacterial material to be used to protect a wound against infection; and
(d) at least one self-adhesive bandage with a bottle of disinfecting wound cleaner and a container of anti-bacterial cream; and
(e) at least one self-adhesive bandage with a bottle of disinfecting wound cleaner and at least one swab.

2. A kit as in claim 1 in which said water is selected from the group consisting of certified spring water and sterile water.

3. A kit as in claim 1 in which said bottle containing said water is a relatively flat and relatively wide-mouth plastic bottle with a removable cap.

4. A kit as in claim 1 in which the package containing said bottle of water includes a relatively stiff card with said bottle held onto said card.

5. A kit as in claim 4 in which said card has a hole shaped to match the outline of said bottle with said bottle fitted into said hole, and packaging material surrounding said card, said bottle and said oral medication package.

6. A kit as in claim 5 in which said packaging material is transparent and extends around both the front and the back sides of said card.

7. A kit as in claim 4 in which said card has a coating of thermoplastic material on at least one surface and said package comprises a plastic covering adhered to said one surface of said card and covering said water bottle and said container of medication materials.

8. A kit as in claim 1 in which said medication materials comprise multiple analgesic pills.

9. A kit as in claim 1 in which said disinfecting wound cleaner is selected from the group consisting of hydrogen peroxide and alcohol.

10. A kit as in claim 1 in which said container of disinfecting wound cleaner is a bottle and the package containing it has a card with said bottle attached to it, said bottle and said card being encased in a transparent plastic covering secured to said card.

11. A kit as in claim 10 in which said card has a hole shaped to match the outline of said bottle with said bottle fitted into said hole, with said covering over both sides of said card.

12. A kit as in claim 1 in which said container of anti-bacterial material is a squeeze tube of anti-bacterial cream.

13. A medication kit comprising, in combination, a support card, a bottle of water, said support card having an opening shaped to the outline of said bottle with said bottle being fitted into said opening, a package of pills to be swallowed with water; and a plastic covering over said support card, said bottle and said package of pills, with said covering holding said bottle in said opening in said support card.

14. A kit as in claim 13 which said card has a thermoplastic surface, with said plastic covering heat-adhered to said surface, and in which said pills comprise analgesics held in at least one sealed envelope.
